(19) United States
(12) Patent Application Publication
(10) Pub. No.: US 2021/0293643 A1
(43) Pub. Date: Sep. 23, 2021

(54) Multi-Modal Fingertip Sensor With Proximity, Contact, And Force Localization Capabilities

(71) Applicant: The Regents of the University of Colorado, a body corporate, Denver, CO (US)

(72) Inventors: Nikolaus Correll, Boulder, CO (US); Radhen Patel, Boulder, CO (US); Jacob Segil, Boulder, CO (US); John Klingner, Boulder, CO (US); Richard F. Weir, Longmont, CO (US)

(73) Assignee: The Regents of the University of Colorado, a body corporate, Denver, CO (US)

(21) Appl. No.: 17/257,715

(22) PCT Filed: Jul. 5, 2019
(86) PCT No.: PCT/US2019/040724

§ 371 Date: Jan. 4, 2021

Related U.S. Application Data
(60) Provisional application No. 62/694,278, filed on Jul. 5, 2018.

(51) Int. Cl. G01L 5/22; B25J 15/00; A61F 2/58

(52) U.S. Cl. CPC G01L 5/226; A61F 2002/7635; A61F 2/586; B25J 15/0009

(57) ABSTRACT

Various embodiments of the present technology generally relate to robotics and prosthetics. More specifically, some embodiments of the present technology relate to multi-modal fingertip sensors with proximity, contact, and for localization capabilities. Various embodiments of the present technology provide for a novel multi-modal tactile sensor which comprises an infrared proximity sensor and a barometric pressure sensor embedded in an elastomer layer. Signals from both of these sensors can be fused to measure proximity (0-10 mm), contact (0N), force (0-50N) and localize impact at five spatial locations and three angles of incidence. Gaussian processes in a regression setting can be used to obtain calibrated force measurements with an R-squared value of 0.99. Supervised machine learning approaches can be used to localize the position and direction of probing with classification accuracies of 96% and 89% respectively.

### CROSS-REFERENCE TO RELATED APPLICATIONS

This application is a national phase of International Application No. PCT/US2019/040724 filed on Jul. 5, 2019, which claims priority to U.S. Provisional Application Ser. No. 62/694,278 filed Jul. 5, 2018, which are incorporated herein by reference in their entireties for all purposes.

### STATEMENT REGARDING FEDERALLY SPONSORED RESEARCH

This invention was made with government support under grant number FA9550-15-1-0238 awarded by the United States Air Force. The government has certain rights in the invention.

### TECHNICAL FIELD

Various embodiments of the present technology generally relate to robotics and prosthetics. More specifically, some embodiments of the present technology relate to multi-modal fingertip sensors with proximity, contact, and force localization capabilities.

### BACKGROUND

Traditional tactile sensors in both the robotics and the prosthetics fields still have many barriers for these sensors to be integrated into self-contained prosthetic hands. In robotics, contact information is useful for a variety of grasping-related tasks such as object identification through haptic exploration/palpation and object manipulation that involves gentle interaction. Proximity information is used primarily for the pre-grasp improvement, reactive grasping, and point-cloud construction of objects. Dynamic force patterns are useful in detecting slip and other such disturbances from the grasped objects as well as for providing sensory feedback to users of prosthetic devices. This in turn informs about the grasp stability associated with an object and allows reactions to unpredicted disturbances. The ability to estimate the position and orientation of the object in hand is an important skill for effective object manipulation.

There are a number of challenges and inefficiencies created in traditional robotic and prosthetic sensors. For example, traditional tactile sensors are unable to detect spatial location of loads and angles of incidence of the force and cannot detect zero force contact/release events. The ability to pre-shape the prehensor in advance of making contact with the object is not possible without proximity information. Thus, it can be difficult to create biomimetic sensory-feedback paradigms like Discrete Event Sensory Control (DESC). It is with respect to these and other problems that embodiments of the present invention have been made.

### SUMMARY

This Summary is provided to introduce a selection of concepts in a simplified form that are further described below in the Detailed Description. This Summary is not intended to identify key features or essential features of the claimed subject matter, nor is it intended to be used as an aid in determining the scope of the claimed matter.

Various embodiments of the present technology generally relate to robotics and prosthetics. More specifically, some embodiments of the present technology relate to multi-modal fingertip sensors with proximity, contact, and force localization capabilities. In some embodiments, a fingertip sensor can include a proximity sensor, a pressure sensor, a circuit with various digital electronics, and a viscoelastic compressible material, and/or other components. The proximity sensor (e.g., infrared emitter-detector) can be used to detect distance from the proximity sensor to an object and produce a proximity signal and detect initial contact. The pressure sensor (e.g., barometer) can be to detect contact with the object and produce a pressure signal indicative of the force being applied. The pressure sensor (e.g., barometer) may provide new readings at a much slower rate than the proximity sensor. For example, the pressure sensor in some embodiments may only provide a new reading every half second while the IR can be sampled up to 1 KHz. The circuit with digital electronics can be configured to receive the proximity signal from the proximity sensor and the pressure signal from the pressure sensor to identify spatial position and angular orientation of the object relative to the fingertip sensor. The viscoelastic compressible material can enclose the proximity sensor, the pressure sensor, and the circuit.

Embodiments of the present invention also include computer-readable storage media containing sets of instructions to cause one or more processors to perform the methods, variations of the methods, and other operations described herein.

While multiple embodiments are disclosed, still other embodiments of the present invention will become apparent to those skilled in the art from the following Detailed Description, which shows and describes illustrative embodiments of the invention. As will be realized, the invention is capable of modifications in various aspects, all without departing from the scope of the present invention. Accordingly, the drawings and Detailed Description are to be regarded as illustrative in nature and not restrictive.

### BRIEF DESCRIPTION OF THE DRAWINGS

Embodiments of the present technology will be described and explained through the use of the accompanying drawings.

FIG. 1 illustrates an example of a hand with multi-modal tactile sensors at each finger that may be used in accordance with some embodiments of the present technology.

FIG. 2 illustrates an example of various components of a fingertip sensor that may be used in accordance with some embodiments of the present technology.

FIG. 3 illustrates an example of a digit of a prosthetic hand or robotic hand that may be used in accordance with some embodiments of the present technology.

FIG. 4 illustrates an example of a portion of a thumb into which the fingertip sensors may be integrated in accordance with some embodiments of the present technology.

FIG. 5 illustrates tested locations of spatial positions (left) and angular orientations (right) according to one or more embodiments of the present technology.

FIG. 6 is a block diagram showing various components of a fingertip that may be used in some embodiments of the present technology.

FIG. 7 is a block diagram showing various components of a centerboard that may be used in some embodiments of the present technology.

FIG. 8 illustrates an example of a sensor response when a small piece of cotton is dropped onto the sensor and pressed according to one or more embodiments of the present technology.

FIGS. 9A-9C illustrate examples of multimodal fingertip readings for a 30N load at five spatial locations where each curve is an average of ten contact events according to one or more embodiments of the present technology.

FIGS. 10A-10C show a Gaussian process regression fit for barometer sensor values, infrared sensor values, and combined, to force in Newtons in accordance with some embodiments of the present technology.

FIG. 11 is flowchart illustrating a set of operations for producing force, proximity, and contact signals in accordance with one or more embodiments of the present technology.

FIG. 12 is a flowchart illustrating a set of operations for identifying a contact event with an object in accordance with some embodiments of the present technology.

FIG. 13 is a block diagram illustrating integration of a machine learning engine in according to various embodiments of the present technology.

FIG. 14 is a flowchart illustrating a set of operations for generating a biomimetic response in accordance with various embodiments of the present technology.

FIG. 15 is a block diagram illustrating a set of components that may be used to control a prosthetic hand or a robotic hand in accordance with various embodiments of the present technology.

FIG. 16 is a state flow diagram illustrating an example of various operational states of a prosthetic hand or a robotic hand in accordance with various embodiments of the present technology.

FIG. 17 is a plot the displacement of an object when using a proximity detection controller on or off that may be used in accordance with various embodiments of the present technology.

The drawings have not necessarily been drawn to scale. Similarly, some components and/or operations may be separated into different blocks or combined into a single block for the purposes of discussion of some of the embodiments of the present technology. Moreover, while the technology is amenable to various modifications and alternative forms, specific embodiments have been shown by way of example in the drawings and are described in detail below. The intention, however, is not to limit the technology to the particular embodiments described. On the contrary, the technology is intended to cover all modifications, equivalents, and alternatives falling within the scope of the technology as defined by the appended claims.

### DETAILED DESCRIPTION

Various embodiments of the present technology generally relate to robotics and prosthetics. More specifically, some embodiments of the present technology relate to multi-modal fingertip sensors with proximity, contact, and force localization capabilities. Numerous tactile sensors have been designed with application to both robotics and prosthetics. However, many barriers remain for these sensors to be integrated into self-contained prosthetic hands. Some of these barriers include the digital communication systems, the multiplexing of multiple sensors, and the wiring of the sensors throughout the device. Simple off-the-shelf pressure sensors (e.g., FlexiForce, Tekscan Inc. South Boston) are well used but lack the ability to detect spatial location of loads and angles of incidence of the force. None of the traditional sensors can detect zero force contact/release events which are important signals for the recreation of biomimetic sensory-feedback paradigms like Discrete Event Sensory Control (DESC) as well as the proximity of objects with respect to the prehensor.

In robotics, contact information is useful for a variety of grasping-related tasks such as object identification through haptic exploration/palpation and object manipulation that involves gentle interaction. Proximity information is used primarily for the pre-grasp improvement, reactive grasping, and point-cloud construction of objects. Dynamic force patterns are useful in detecting slip and other such disturbances from the grasped objects. This in turn informs about the grasp stability associated with an object and allows reactions to unpredicted disturbances. The ability to estimate the position and orientation of the object in hand is an important skill for effective object manipulation. However, there are only few sensors that combine all of this information into a single package and few if any have been effectively translated to address the unique challenges of prosthetic limb design.

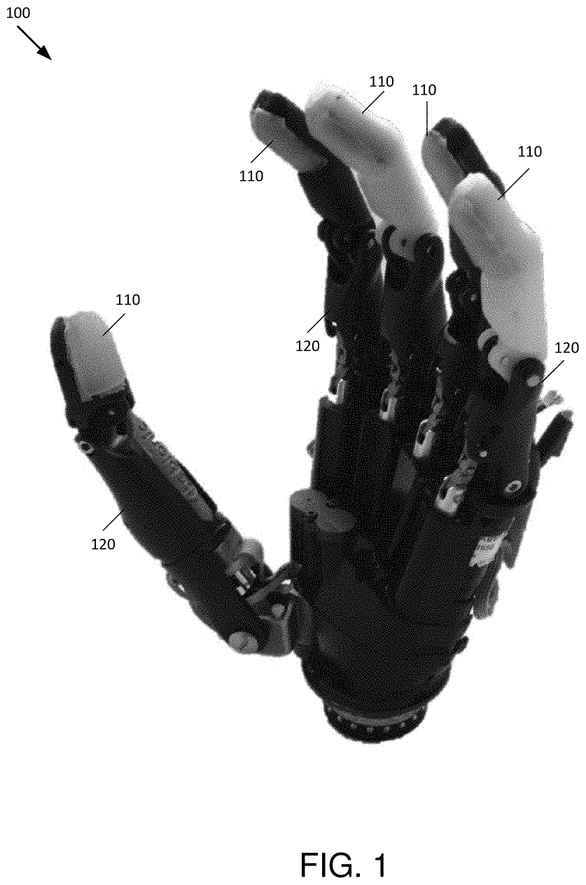

In contrast, various embodiments of the present technology include a sensor (e.g., for a prosthetic or robotic fingertip) which integrates both an infrared emitter-detector and barometer to form a proximity, contact, and force sensor (see, e.g., FIG. 1). In accordance with various embodiments, IC sensors are integrated into a prosthetic finger and over molded with an elastomer to create a robust contact surface for the prostheses. Standard I²C communication between sensors and prosthetic hand controller can be used and can ensure stable and reliable communication. This multi-modal sensory information (proximity, contact, and pressure), when synthesized, provides rich data to perform sensory fusion to derive additional information not available from each sensor independently. The resulting multimodal fingertip sensors provide zero-force contact sensing, linear force readings (e.g., from 0N to 50N), and the ability to classify multiple spatial locations (e.g., five spatial locations) and multiple angles of incidence (e.g., three angles of incidence) in a self-contained fingertip sensor.

Various embodiments of the present technology provide for a novel multi-modal tactile sensor which comprises an infrared proximity sensor and a barometric pressure sensor embedded in an elastomer layer. Signals from both of these sensor can be fused to measure proximity (0-10 mm), contact (0N) and force (0-50N) and to localize impact at five spatial locations and three angles of incidence. Gaussian processes in a regression setting can be used to obtain calibrated force measurements with an R-squared value of 0.99. Supervised machine learning approaches can be used to localize the position and direction of probing with classification accuracies of 96% and 89% respectively. Preliminary experiments show the complimenting nature of both sensors that lead to several sensing modalities that no sensor can provide on its own with potential use in prosthetics and robotics.

Various embodiments of the present technology provide for a wide range of technical effects, advantages, and/or improvements to computing systems and components. For example, various embodiments include one or more of the following technical effects, advantages, and/or improvements: 1) tactile sensor including multiple sensor modalities allowing simulation of biomimetic responses; 2) integrated use of machine learning to identify contact, forces, and angles of interactions with an object; 3) use of tactile sensors to provide pre-shaping of an artificial hand to reduce crushing, tipping, or other unwanted interactions with an object; 4) use of unconventional and non-routine computer operations to improve grasping interactions; 5) cross-platform integration of machine learning to more efficiently operate artificial hands and limbs; 6) changing the manner in which an artificial hand interacts with environmental situations; 7) changing the manner in which an artificial hand reacts to user interactions and feedback; and/or 8) improving sensory feedback signals used to restore sensation in prosthetic device users

In the following description, for the purposes of explanation, numerous specific details are set forth in order to provide a thorough understanding of embodiments of the present technology. It will be apparent, however, to one skilled in the art that embodiments of the present technology may be practiced without some of these specific details.

The techniques introduced here can be embodied as special-purpose hardware (e.g., circuitry), as programmable circuitry appropriately programmed with software and/or firmware, or as a combination of special-purpose and programmable circuitry. Hence, embodiments may include a machine-readable medium having stored thereon instructions which may be used to program a computer (or other electronic devices) to perform a process. The machine-readable medium may include, but is not limited to, floppy diskettes, optical disks, compact disc read-only memories (CD-ROMs), magneto-optical disks, ROMs, random access memories (RAMs), erasable programmable read-only memories (EPROMs), electrically erasable programmable read-only memories (EEPROMs), magnetic or optical cards, flash memory, or other type of media/machine-readable medium suitable for storing electronic instructions.

The phrases “in some embodiments,” “according to some embodiments,” “in the embodiments shown,” “in other embodiments,” and the like generally mean the particular feature, structure, or characteristic following the phrase is included in at least one implementation of the present technology, and may be included in more than one implementation. In addition, such phrases do not necessarily refer to the same embodiments or different embodiments.

FIG. 1 illustrates an example of hand 100 with multi-modal tactile sensors 110 at each finger 120 in accordance with some embodiments of the present technology may be utilized. As integrated into FIG. 1, one or more of the tactile sensors 110 can be integrated into the prehensor (e.g., robotic or prosthetic hand). In accordance with various embodiments, the fingertip or tactile sensor can use two sensors: a barometric pressure sensor such as a MEMS-based barometric pressure sensor (e.g., MS5637-02BA03) and an infrared proximity sensor (e.g., VCNL4010). Assembly of the sensor can involve multiple steps, though various embodiments of the present technology are not limited to the following possible combination and order of steps.

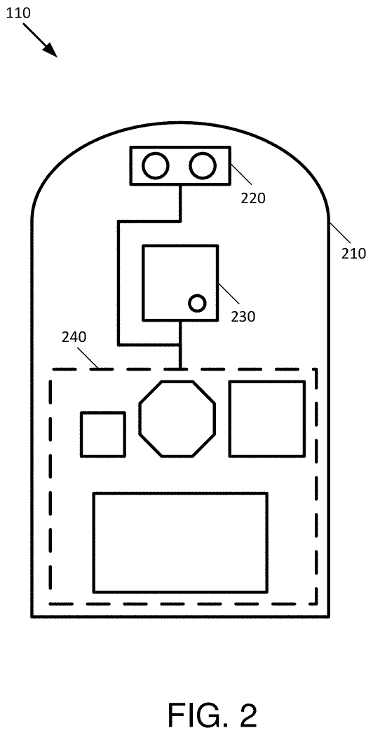

FIG. 2 illustrates an example of various components of a fingertip sensor 110 that may be used in some embodiments of the present technology. As illustrated in FIG. 2, the multi-modal tactile sensors 110 can be arranged on a printed circuit board (PCB) 210, or other substrate, and can be positioned along a midline of a prosthetic or robotic fingertip (e.g., as illustrated in FIG. 1) or other position based on likely contact points for the digits or thumb. The combination of proximity sensor (e.g., IR sensor) 220 and pressure sensor (e.g., barometer) 230 along with substrate 240 can be referred to as the “sensor assembly.” A cavity for the sensor assembly can be formed in a finger of a prosthetic or robotic hand or other prehensor. For instance, a cavity can be formed in fingers of the Bebionic v2 hand (RSL Steeper) (see FIG. 1).

One or more of these fingers 120 can be formed via 3D printing around the sensor assembly, or the finger(s) can be 3D printed to allow for the sensor assembly to be inserted into the 3D printed finger after creation. Alternatively, an elastomer, such as liquid silicon polymer (e.g., Dragon skin 10), can be poured into a mold containing the sensor assembly such that the finger is “overmolded” over the sensor assembly. The elastomer preferably has low viscosity when poured into molds and mechanical robustness post curing. In some embodiments, a vacuum can be applied before pouring the elastomer into the mold to completely remove air from the polymer.

In accordance with various embodiments, tactile sensor 110 can include a logic circuit (e.g., PCB with a logic circuit printed thereon) that can be used to multiplex the sensor assembly's communication (e.g., using Inter-Integrated Circuit (I²C) Protocol) signals for access by a host computing device. The host computer can be separate from the prosthetic or robot or can be incorporated into the prosthetic or robot (e.g., a central controller board). For instance, the host computer can be worn on other anatomy of a user of the prosthetic. A microcontroller (e.g., Arduino) can be used to perform the multiplexing. In some embodiments, the multiplexing can include two signals per finger (one from the pressure sensor and one from the proximity sensor), such that the total number of signals to be multiplexed is n*2, where n is a number of fingers). The microcontroller firmware can perform the proximity calculation for the proximity sensor as well as the calibration and temperature compensation for the pressure sensor (e.g., using algorithms provided by the sensor manufacturer). The firmware can then send calibrated proximity and pressure data to the laptop computer through a serial USB interface. Some embodiments use a custom LabView (National Instruments Inc.) program to visualize real-time signals from the sensor assembly and can store data off-line for processing and analysis.

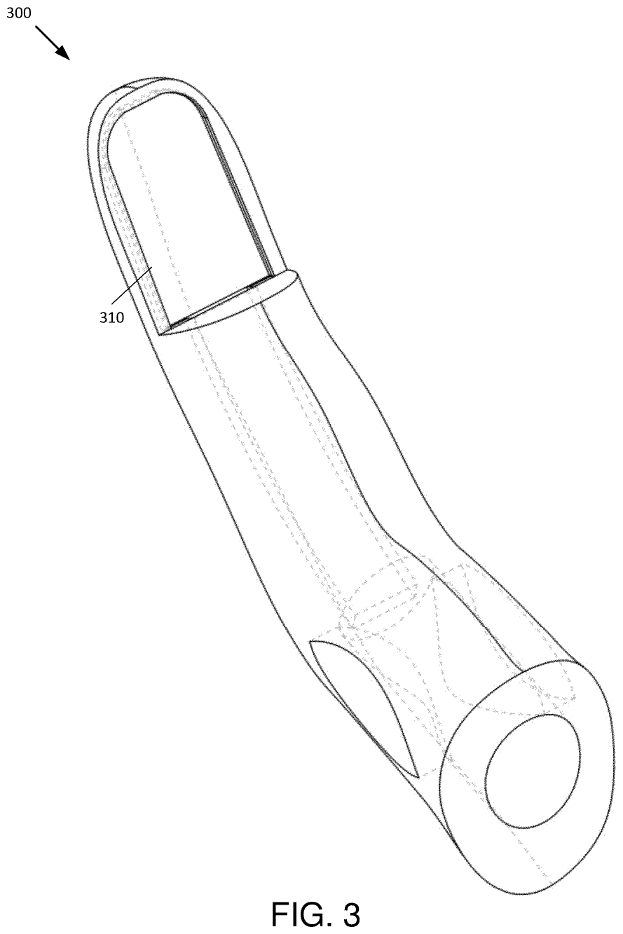

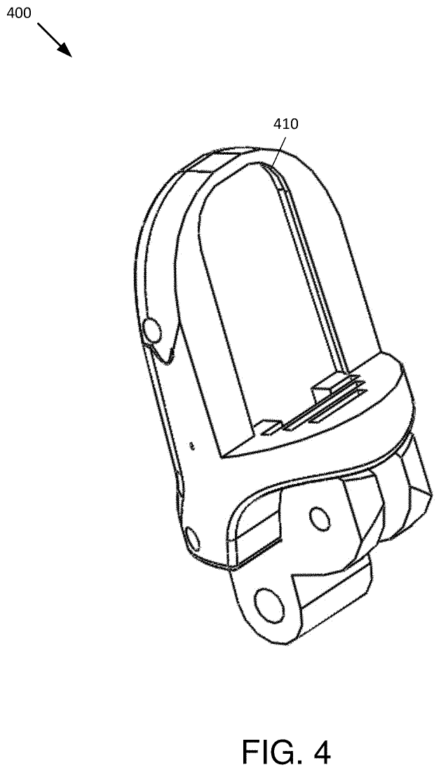

FIG. 3 illustrates an example of a digit 300 of a prosthetic hand or robotic hand that may be used in accordance with some embodiments of the present technology. FIG. 4 illustrates an example of a portion of a thumb 400 in which the fingertip sensors may be integrated into in accordance with some embodiments of the present technology. As can be seen in FIGS. 3 and 4, a cavity or recess 310 or 410 can be integrally formed within digit 300 or thumb 400 and formed to allow the sensor assembly to be securely affixed (e.g., with a press fit, snaps, or other mechanism) into the cavity or recess.

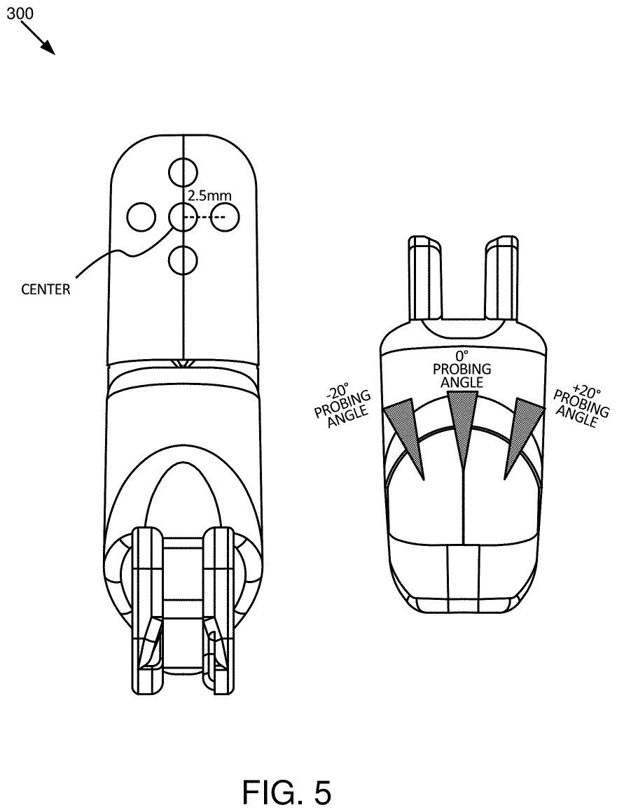

To experimentally characterize the performance of the sensors, multiple fingertip sensors were fabricated and tested. An Instron material testing machine (MTS Insight II—Low capacity: 2 kN maximum) applied calibrated loads to various spatial positions and angles of incidence on the fingertip as detailed below. The loads were applied using a probe with a flat circular tip (15 mm diameter) and monitored using a 250N load cell (model: M569326-06, sensitivity: 2.016 mV/V). The MTS machine applied prescribed loads ranging from 1N to 50N) at a rate of 1 mm/s with a sampling rate of 16 Hz. Additional fingertip “pillows” were prototyped in order to locate the fingertip sensor in the prescribed spatial and angular orientations with respect to the probe. The spatial dataset measured contact events at the center, 2.5 mm distally, 2.5 mm proximally, 2.5 mm medially, and 2.5 laterally and the angular orientation dataset measured contact events at 0 degree, 20 degree, and −20 degree angles. FIG. 5 illustrates the tested locations of spatial positions (left) and angular orientations (right) according to one or more embodiments of the present technology.

These spatial and angular conditions were chosen in order to span the entire range of the detectable volume of the fingertip sensor. The center location was defined as directly above the midpoint of the PCB. The angular orientations were defined with respect to the normal vector of the PCB. In each condition, a sequence of 10 contact events at each maximum load took place. Each contact event was separated by a 1 second delay. The maximum loads tested were 1 N, 5N, 30N, and 50N. These loads were chosen to span a typical range of loads seen by fingers in everyday use.

The sensor fusion study followed the following procedure: The direction of probing angle was fixed to 0 degrees to obtain the mapping from the analog proximity and pressure readings to true force in Newtons. Ten dynamic loading and unloading cycles were performed on the finger using the same Instron machine described above. To generalize these loading and unloading cycles to everyday forces that the sensor would experience, various embodiments perform this test with multiple maximum load forces (1, 5, and 50 N). Note that the finger 300 and the probing location are kept constant for this calibration. In total, 10 curves for each maximum load force from the barometer sensor, IR sensor, and the load cell for a total of 90 curves (10×3×3) were created.

To collect data for classifying the direction of probing, 10 dynamic loading and unloading cycles were performed with the Instron machine for the maximum peak forces of 1, 5, 30, and 50 N at 0, 20, and −20 degrees of probing direction. Custom-made 3D-printed pillows were used for the finger 300 that align it at various angles with respect to the probe. In total, 120 combined loading and unloading curves were produced.

To determine the spatial location of impact on the finger, the data were collected by probing the finger 300 at different locations with respect to the center of the finger 300 (see, e.g., FIG. 5). Making use of the custom-made 3D-printed pillows to align/offset the finger with respect to the center of the probe. The data collection procedure consisted of 10 dynamic trials of loading and unloading for each of the maximum forces of 1, 5, 30, and 50 N for five spatial locations with respect to the barometer. The data are segmented into a single combination of loading and unloading curves summing to a total of 200 curves (10×4×5).

The calibration of multi-modal fingertip data to measure force is non-trivial. The combined signals from the fingertip vary based on the position and orientation of contact. Therefore, it is challenging to estimate a single function with a fixed number of parameters that will map the raw barometer and IR readings to true force in Newtons. To help solve this problem, various embodiments of the present technology use a Gaussian process (GP) in a regression setting to map the sensor input to a calibrated force measurement.

The GP approach is a non-parametric approach in that it finds a distribution over the possible functions f(x) that are consistent with the observed data. In a regression setting, one can aim at finding the function with y=f(x)+E with y being the observations, x a set of independent variables, and E being an error term. A GP is defined by a mean function m(x) and covariance function k(x,x^{t}), otherwise known as a kernel function. GP defines a prior over the possible functions, which can be converted to posterior once data is available. In other words, there are some known parameters x for which there is some observed outcome f(x). Suppose there are some points x* for which one would like to estimate f(x*).

An estimate of the conditional probability p(f*|x,x*,f) on the assumption that the functions f and f* are drawn from a joint distribution defined by the GP. A specific advantage of Gaussian processes in our case is that they are computationally affordable on small datasets and have a well-tuned smoothing property.

Various embodiments frame the problem of localizing external loads on the finger into two separate supervised-learning problems: 1) classification of the spatial location of load, and 2) classification of the angle of incidence of the force at 0 degree, 20 degree, and −20 degree angles (see, e.g., FIG. 5). The organization of the machine learning methods here was used as a proof of concept for a more sophisticated algorithm that could classify both spatial and angular orientation in real time.

Support vector machine (SVM), k-nearest neighbor (kNN), dynamic time warping (DTW), naive Bayes, and so on are very popular due to their high computational efficiency and high resistance to noise. However, it is inherently difficult to design good features that can capture intrinsic properties embedded in various time series data. Several deep learning frameworks are better in such cases as they do not need any handcrafted features by people, instead they can learn a hierarchical feature representation from raw data automatically. To compare these two supervised learning frameworks, an SVM classifier and a convolutional neural network (CNN) was trained for each of the supervised learning problems.

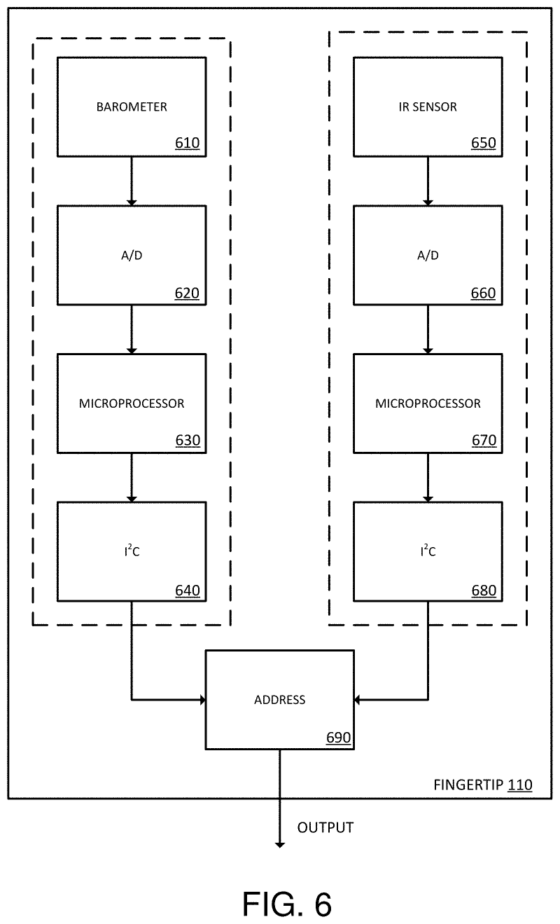

FIG. 6 is a block diagram showing various components of a fingertip 110 that maybe used in some embodiments of the present technology. As shown in FIG. 6, fingertip 110 may include barometer or pressure sensor 610, analog to digital (A/D) converter 620, microprocessor 630, I²C communications port 640, IR or distance sensor 650, A/D converter 660, microprocessor 670, I²C communications port 680, and address module 690. Some embodiments may include additional components not shown in FIG. 6. Examples include, but are not limited to, a memory (e.g., volatile memory and/or nonvolatile memory), a power supply (e.g., battery), and the like.

Pressure sensor 610 can provide a measurement of the pressure within the fingertip sensor. For example, pressure sensor 610 may provide a linear measurement of the applied force after a minimum range has been crossed. In some embodiments, pressure sensor 610 may be a single element or an array of pressure sensors to provide an array of measurements. Pressure sensor 610 may be a barometric pressure sensor that flexes inward and causes an increase in atmospheric pressure within the sensor. This change in pressure can be sensed by the device's internal barometer and translated to an analog output signal (e.g., a voltage signal). The analog output signal can then be converted to a digital signal using A/D converter 620 which microprocessor 630 can map into an estimate of the touch force on the fingertip. I²C communications port 640 allows multiple pressure sensors to communicate via address bus 690 with other integrated circuits such as a central controller board (not shown). In some embodiments, data may be transferred at a rate between 100 kHz to 400 kHz.

IR or distance sensor 650 can be an infrared (IR) emitter-detector to detect the distance between the sensor and the object. The measurement can be provided as an analog output which A/D converter 660 can convert to a digital signal which microprocessor 670 can use to create an estimate of the distance. I²C communications module 680 allows the output of microprocessor 670 to be communicated to other integrated circuits or controllers (e.g., a central controller board.

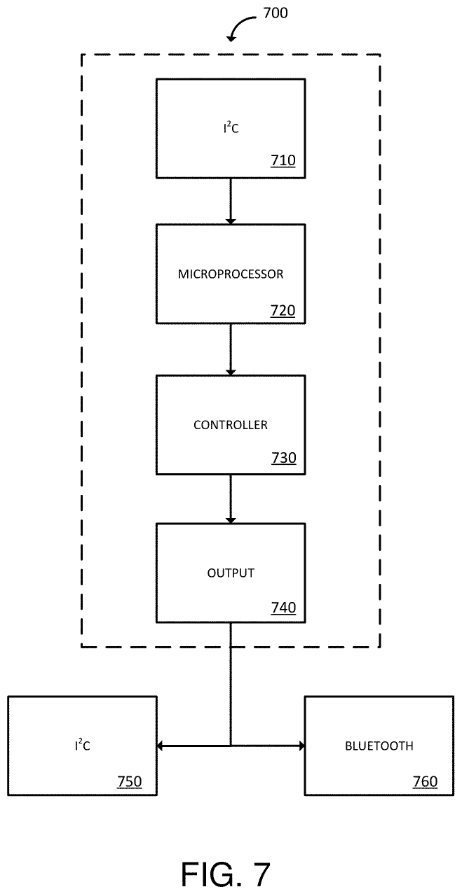

FIG. 7 is a block diagram showing various components of a centerboard 700 that may be used in some embodiments of the present technology. Centerboard 700 (or central controller) may be located within a robotic or prosthetic hand or located externally to the hand. Centerboard 700 can be configured to receive an array of output from one or more fingertip sensors. For example, I²C communication channel 710 can receive pressure and distance measurements from each fingertip sensor. Microprocessor 720 can take this information and determine, e.g., using controller 730, one or more outputs 740 providing control actions for the actuators within the prosthetic or robotic hand. Outputs 740 may be transmitted to motors using I²C communication channel 750, Bluetooth communication channel 760, and/or other communication channel.

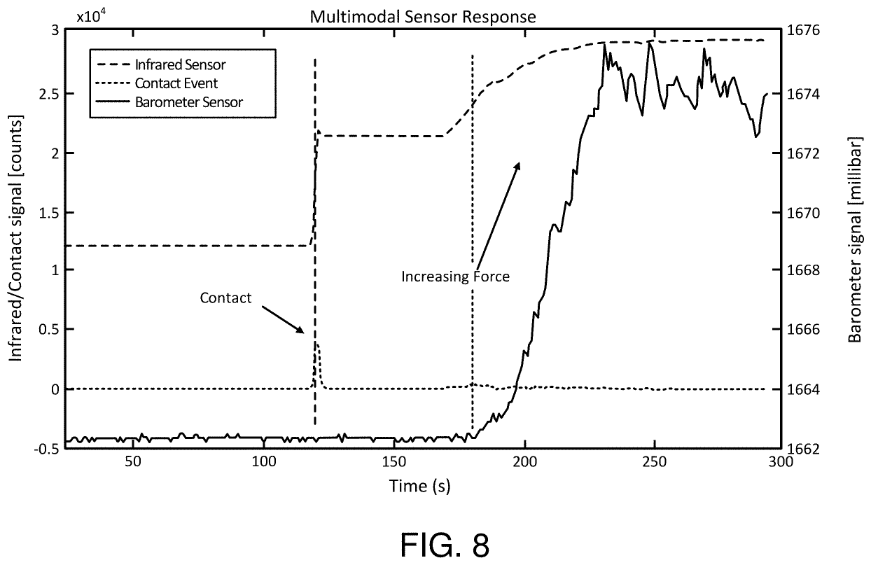

FIG. 8 illustrates an example of a sensor response when a small piece of cotton is dropped onto the sensor and pressed according to one or more embodiments of the present technology. The multiple sensing modalities of the sensor are depicted in the FIG. 8. A small piece of cotton is dropped from a fixed height onto the sensor and gently pressed. The contact detection is clearly visible as a small peak in the contact event curve. The cotton is then gently pressed against the sensor. This change in the force is picked up by the barometer in a linear manner.

Cotton was chosen because of its light weight and to show that the infrared sensor can detect contact forces close to 0N that the barometer cannot measure. The proximity signal includes some nonlinear elements which are visible in the curve at the time force is applied on the cotton. In the embodiments illustrated in FIG. 8, the contact signal was derived by passing the raw infrared signal through a high-pass filter such as a first order Butterworth high-pass filter. The barometer provides a linear measurement of the pressure within the fingertip sensor which is stable across all loads (tested up to 50N). These signals are representative of the proximity, contact, and pressure readings across all forces at the centered position, but vary greatly with variations in the spatial position and/or angular orientation.

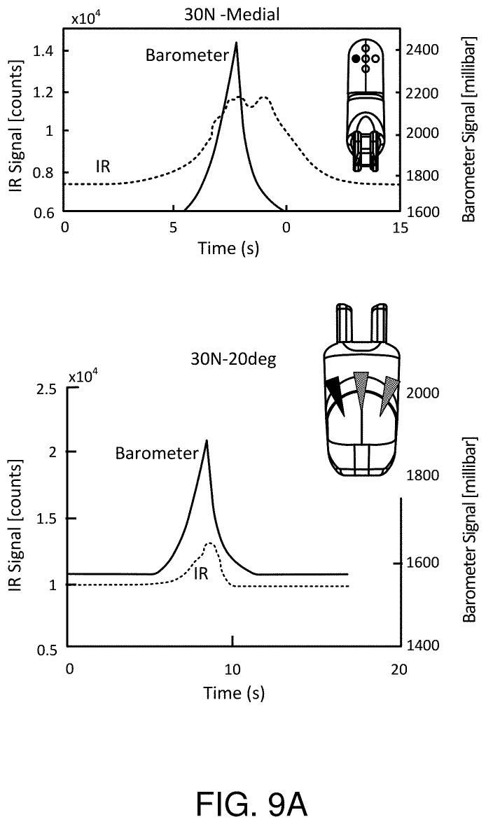

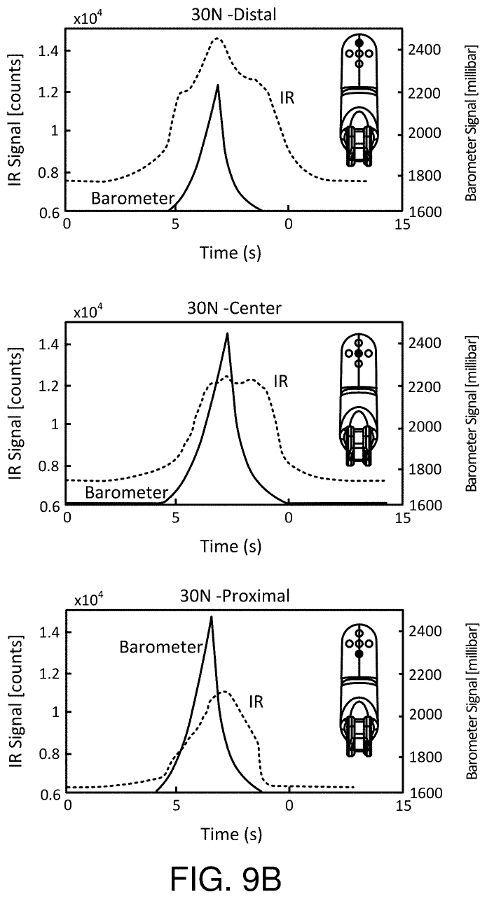

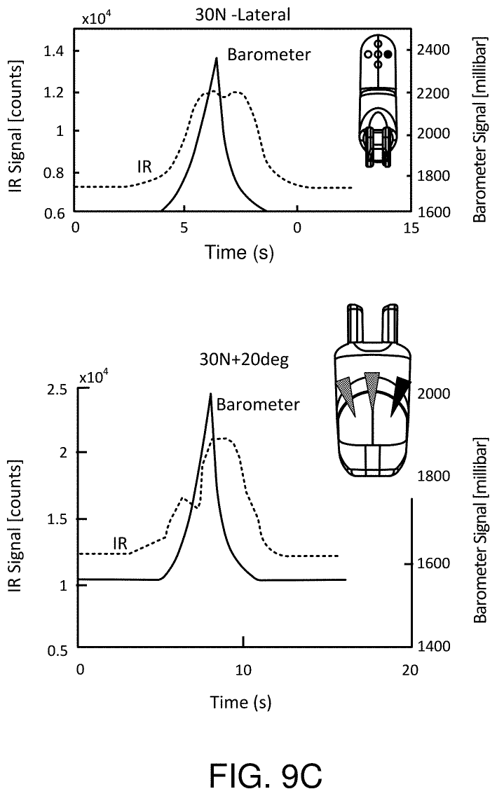

FIGS. 9A-9C illustrate examples of multimodal fingertip readings for a 30N load at five spatial locations where each curve is an average of ten contact events according to one or more embodiments of the present technology. The responses of the barometer and infrared proximity (IR) sensor to the applied force in Newtons at any spatial location on the finger are distinctively different. FIGS. 9A-9C show the response of both sensors at a 30N load and zero degree probing angle for all spatial conditions.

FIGS. 9A-9C also show the response of both sensors at a 50N load across all angles of incidence. The barometer shows a linear behavior to applied force after its minimum range has been crossed, whereas the IR sensor shows a nonlinear behavior while being sensitive at a range below that of the barometer. Their behavior is repeatable over a fixed location (each curve is an average of 10 contact events) on the finger over multiple days but varies in an unpredictable manner across those positions on the finger. These variations are more dramatic for the IR sensor compared to the barometer sensor.

To study the relationship between the proximity and pressure readings to true force, various embodiments fixed the direction of probing angle to 0 degrees. Ten loading and unloading cycles were performed on the finger using the same Instron machine described above. These loading and unloading cycles were generalized to everyday forces that the sensor would experience by performing with multiple maximum load forces (1 N, 5N, and 50N). Note that the finger and the probing location is kept constant for this experiment. This gives ten peaks for each maximum load force from the barometer sensor, IR sensor and the load cell for a total of 90 peaks (10×3×3).

Some embodiments may include data preprocessing steps including passing the raw sensor signals (proximity and pressure) through a low-pass filter to remove unwanted noise from the signal. To segment out an individual curve consisting of loading and unloading cycles at a particular maximum peak load force, the peaks were first located from each contact. After locating the peaks, a window of 180 samples (90 samples on each side of the peak) was taken and segment out the individual loading and unloading curves. Individual peaks were then concatenated from each sensor at peak load force of 1 N, 5N, and 50N into a single array. This gives a 3×10 set of data: three sensors (two on the finger and the external force sensor) and ten measured contact events.

The kernel of the Gaussian process was trained by providing it a set of inputs Xtrain and targets Ytrain (normalized). Inputs correspond to concatenated raw IR and barometer values, and targets correspond to forces in Newtons from the external load cell. The Gaussian kernel being used is a radial-basis function (RBF) kernel (also known as squared-exponential kernel) implemented in the Scikit-learn library. After the kernel has learned the relationships within the data (Xtrain and Ytrain) the kernel is presented with the testing dataset to predict the labels Ypred given the Xtest. The accuracy of the fit is determined using the rootmean-square error (RMSE) and R-squared (R2) score.

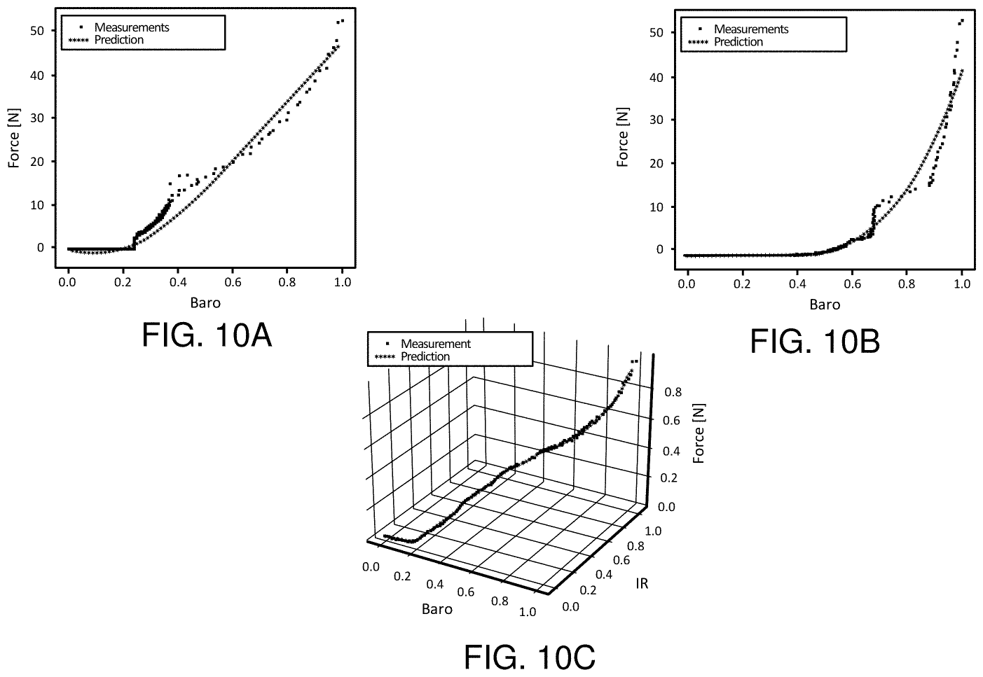

FIGS. 10A-10C show a Gaussian process regression fit for barometer sensor values, infrared sensor values, and combined, to force in Newtons in accordance with some embodiments of the present technology. In FIGS. 10A-10C individual curve fits are presented for barometer and IR readings (before concatenating them) and the fit in 3D after concatenating them and learning the kernel. Note that the kernel parameters are the same for all three fits and are experimentally calculated to minimize error in the 3D plot. The RMSE and R2 score of the three fits are shown in Table I.

TABLE I
Root mean square and R2 error measure for
curve fitting (ref. FIGS. 10A-10C).

| | Barometer | IR | Both |
|---|---|---|---|
| RMSE | 0.02 | 0.03 | 0.01 |
| R2 | 0.98 | 0.96 | 0.99 |

The interaction between the elastomer shell enclosing the sensors and the sensors themselves is difficult to study. This interaction leads to proximity and pressure signals of varying nature from the sensor when it is impacted from different directions and at different locations. To localize impact on such a dynamic sensor various embodiments can break down the problem into two smaller sub-problems. First, the method can identify the angular direction of probing, and second, the spatial location of impact with respect to the center of the fingertip. This can be framed as a classification problem in a supervised learning framework, and train a SVM and a CNN for each of the subproblems: 1) Probing direction; and 2) Spatial location.

To collect data for the probing direction classification a plurality (e.g., 10) of loading and unloading cycles were performed with the Instron machine for a plurality of maximum peak forces (e.g., 1 N, 5N, 30N, and 50N) and at a plurality of probing directions (e.g., 0, 20, and −20 degrees of probing direction). Some embodiments may use custom-made 3D-printed pillows for the finger that align it at various angles with respect to the probe. Assuming 10 loading and unloading cycles, 5 maximum peak forces, and three different probing directions, 120 combined loading and unloading curves were produced. Data preprocessing steps can include locating the peaks from every data collection trial. After locating the peaks, some embodiments can take a window size of X samples (e.g., 150), with half the samples on each side of the peak, and segment out the individual loading and unloading curves. Some embodiments then standardize the individual loading and unloading curves to have zero mean and one variance.

Some embodiments can use SVM as a baseline classifier since the amount of data collected for classification is small. An advantage of such a model is that fewer parameters need to be learned and the user has greater control over the model itself. A couple of variations of the barometer and IR sensor values can be explored to create features for the SVM. The most promising feature was the ratio of the IR and barometer values which gave us a significant rise in testing accuracy. Some embodiments also included the data points of maximum force and minimum force from the sensor into our feature vector. In some embodiments, a polynomial kernel can be used with a penalty factor of C=1. In order to avoid over fitting of our models to the data, cross validation was performed on all of the models described below. The accuracy obtained after 6-fold cross validation is shown in Table II.

TABLE II
Accuracies for probing angle classification.

| Trial | 1st | 2nd | 3rd | 4th | 5th | 6th |
|---|---|---|---|---|---|---|
| SVM | 95% | 95% | 81% | 90% | 83% | 89% |
| | Average accuracy: 89% (+/−5.4%) | | | | | |
| CNN | 86% | 76% | 86% | 81% | 89% | 77% |

Average accuracy: 83% (+/−4.6%)

In addition to this, some embodiments may train a small neural network to classify probing direction. Since convolution inherently captures the relation between the signals it is convolving across, the features ourselves may not have to be hand engineered. The raw data can be fed directly into the network in some embodiments. The network may include two 2D-convolution layers followed by a flattened layer and finally a dense output layer of 3 neurons with softmax activation. The accuracy obtained after 6-fold cross validation on the training and testing dataset is shown in Table II.

To determine the spatial location of impact on the finger some embodiments can use the same supervised learning models described above with different parameters. The data can be collected by probing the finger at different locations with respect to the center of the finger. Custom-made 3D-printed pillows may be used to align/offset the finger with respect to the center of the probe. As mentioned earlier, the signal from the IR sensor has greater variations as compared to the barometer. This variation may be an important factor in achieving the unexpected greater classification accuracy of using both a proximity and pressure sensor as opposed to just a pressure sensor.

The data collection procedure can include Y number of trials (e.g., Y=10) of loading and unloading for each of X number of maximum forces (e.g., 1 N, 5N, 30N, and 50N) for Z spatial locations (e.g., Z=5) with respect to the barometer. The data is segmented into a single combination of loading and unloading curves summing to a total of Y×X×Z (e.g., 10×4×5=200 curves). The data can then again be standardized before feeding it into the models.

The features extracted for training the SVM may be similar to those described previously. A radialbasis function (RBF) kernel with a penalty factor of C=8 was experimentally found to give a mean classification accuracy of 0.959 (+/−0.0354) after 6-fold cross validation (Table III).

The neural network architecture is also the same as described previously, except for an increase in the number of output neurons on the dense layer from 3 to 5, as now there are 5 labels to classify. The number of filters, their size, and kernel parameters were kept constant to compare the results. The accuracy obtained after 6-fold cross validation on the training and testing dataset is shown in Table III.

TABLE III
Accuracies for spatial location classification.

| Trial | 1st | 2nd | 3rd | 4th | 5th | 6th |
|---|---|---|---|---|---|---|
| SVM | 94% | 94% | 97% | 100% | 90% | 100% |
| | Average accuracy: 96% (+/−3.5%) | | | | | |
| CNN | 88.5% | 91% | 97% | 97% | 87% | 93% |

Average accuracy: 92% (+/−3.9%)

The herein disclosed sensor assembly has a variety of applications in robotic/prosthetic grasping and manipulation due to its ability to estimate proximity, contact, force, location, and direction of impact. These signals are important when, for example, the object is to be reoriented in the hand or the object is intended to be used as a tool.

The Gaussian processes method used in some embodiments can enable fusing of the pressure and proximity sensor data to force (e.g., Newtons). For the classification task, SVM outperforms the CNN approach, which is believed to be due to overfitting. Although the numerical values are a good fit, the proposed methods might not generalize over different probing shapes and material since the shape of indentation on the elastomer drives the signals in an unpredictable manner. Even though Gaussian processes regression is the most accurate regression method, it has an exceptionally high computational complexity which prevents its usage for large numbers of samples or learning online. The infrared proximity sensor has a strong dependence on the surface properties (e.g., color, texture, and reflectivity) of an object which can throw off the calibration for objects. However, it is believed that the sensor's multiple sensing modalities may help to mitigate some of the challenges discussed above. The linear behavior of the barometer could help calibrate the sensor against objects with a variety of surface properties, and the nonlinear response of the infrared sensor could be used to identify those surface properties.

Further, there are currently no standardized benchmarks for tactile sensing for robotics/prosthetics and manipulation. Here, system-level performance for specific tasks might become more important than characterization of individual sensor characteristics. At the same time, deep reinforcement learning is emerging as a promising technique to learn task level behaviors. Having shown the ability to identify task relevant patterns in data, these techniques might strongly benefit from multi-modal tactile sensing information such as is provided by the sensor presented here. Similar thinking applies to using the sensor in a myoelectric-prosthetic control context. Various embodiments of the present technology may include a myoelectric interface to detect voluntary muscular contractions from a patient and generate the volitional signal. The volitional signal can be a myoelectric signal collected from electrodes positioned on a limb of a subject.

Various embodiments of the sensor assembly's extended spatial capabilities will provide relevant force feedback to amputees even when an object is not centered against each digit. This fact will provide a better sensor for advanced neural interfaces since one can ensure a reliable source of force feedback during the complex activities of daily life. This is possible due to the effectiveness of these two distinct signals: 1) the reflectance of IR light off a reflecting surface and 2) the change in pressure due to the compression of an elastomer.

Various embodiments provide for a multi-modal fingertip sensor which can include an infrared proximity sensor and a barometer embedded in an elastic polymer. The compact sensors include all of the instrumentation, analog-to-digital conversion, and control circuitry which ensure reliable signal quality using the standard I²C communication protocol. The molded elastomer fingertip surface provides a durable interface to manipulate objects while allowing reliable measurements of those interactions. The fingertip sensor can be mapped to actual loads. For instance, some embodiments characterized the fingertip sensor over loads varying between 1N and 50N, and measured the systems response to loads applied spatially about the center and angled with respect to the normal surface of the fingertip. This characterization encompassed 28 distinct loading scenarios. Some embodiments use a Gaussian processes model to fuse the raw barometer and IR sensor readings to determine the applied force with an R-squared value of 0.99.

Then, the location of loading can be identified using supervised learning methods and obtained a classification accuracy of 96% and 92% using a Support Vector Machine and Convolution Neural Network, respectively. They similarly classified the probing angle and obtained classification accuracies of 89% and 83%, respectively.

These sensors can also be integrated with neural interfaces to provide rich sensory information to upper-limb amputees and robots. The calibrated force signal can provide a reliable tactile signal while the proximity and contact signals can allow for investigations of new sensory paradigms. The proximity signal can be mapped to non-physiological percepts while the contact signal can be utilized in a DESC-based manner. Furthermore, real-time sensor-fusion classification can be implemented. Once accomplished, the spatial and angular information may be relevant to certain neural interfaces and/or may be used in shared control paradigms of the prosthetic limb.

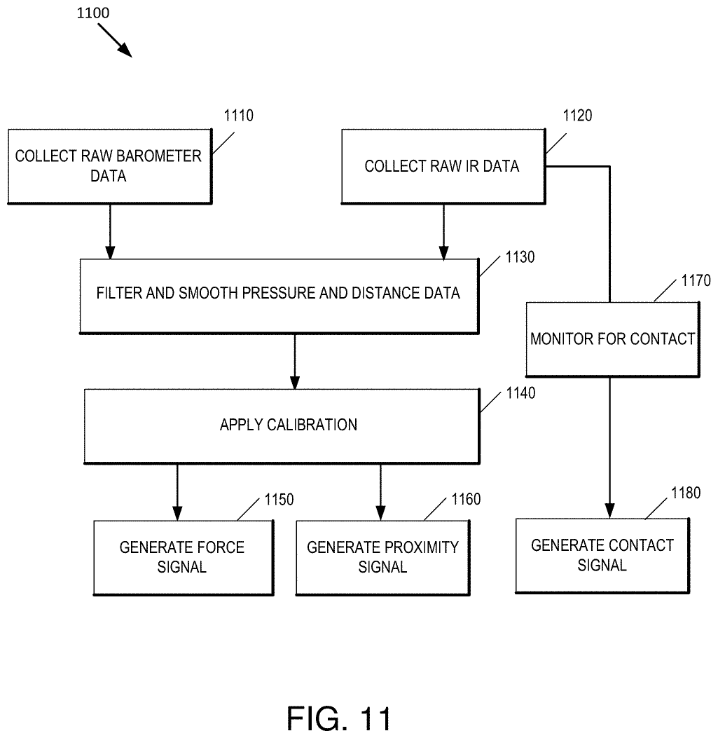

FIG. 11 is flowchart illustrating a set of operations 1100 for producing force, proximity, and contact signals in accordance with one or more embodiments of the present technology. As illustrated in FIG. 11, data collection operation 1110 collects the raw pressure (e.g., barometer) data. During distance collection operation 1120, raw distance data (e.g., IR data) is collected. The raw data can be filtered and smoothed using filtering operation 1130. Calibration operation 1140 can apply any calibration offsets or modifications to the filtered data to provide force signal 1150 and proximity signal 1160 which can be used to make decisions for controlling digits of the prehensor or hand. The raw IR data collected by distance collection operation 1120 can be used by contact detection operation 1170 to identify contact event. For example, in some embodiments, the derivative of the IR data can be computed and a detected spike can be used to identify contact with the fingertip which signal generation operation 1180 uses to produce a contact signal.

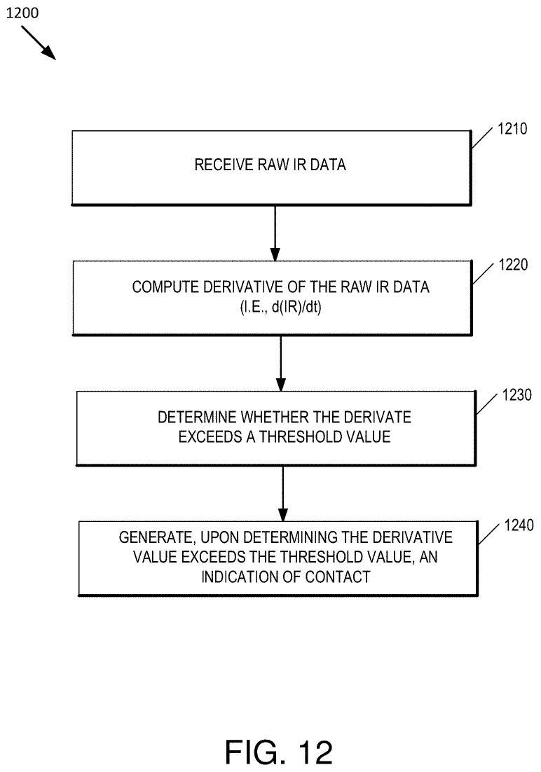

FIG. 12 is a flowchart illustrating a set of operations 1200 for identifying a contact event with an object in accordance with some embodiments of the present technology. As illustrated in FIG. 12, receiving operation 1210 receives the raw distance signal (e.g., IR data signal). Computation operation 1220, computes the derivative of the raw distance data. Determination operation 1230 determines whether the derivative exceeds a threshold, and generation operation 1240 generates a contact signal in response to a determination that the derivative value exceeds the threshold value.

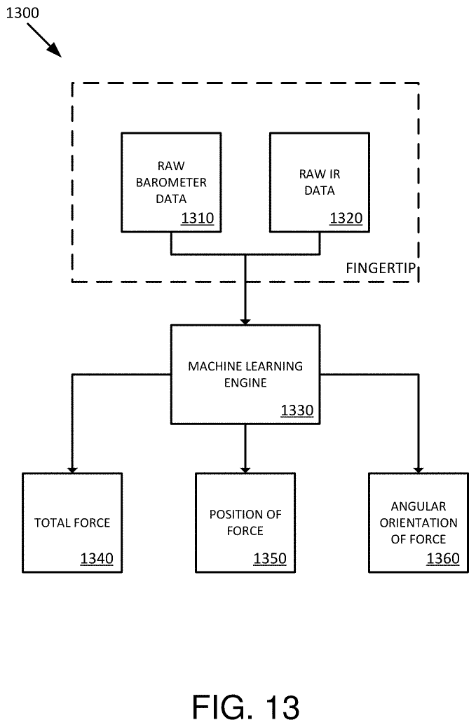

FIG. 13 is a block diagram 1300 illustrating integration of a machine learning engine in according to various embodiments of the present technology. As illustrated in FIG. 13, raw barometer and IR data 1310 and 1320 are collected at one or more fingertips. This data is communicated to machine learning engine 1330 which computes a total force signal 1340, a position of force signal 1350, and an angular orientation of force signal 1360. The machine learning engine 1330 may include a model that is trained offline and outside of the prehensor. The machine learning engine 1330 may include various processors, memory and communication components. In some embodiments, the communications components may be able to receive updated models (e.g., from a cloud-based training engine that analyzes large data sets).

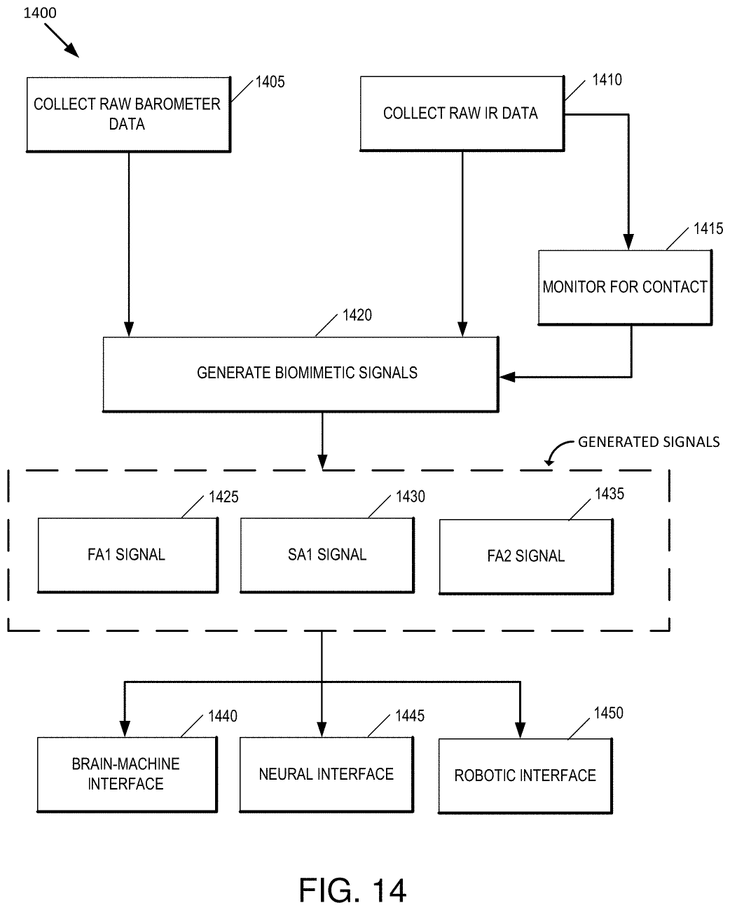

FIG. 14 is a flowchart illustrating a set of operations 1400 for generating a biomimetic response in accordance with various embodiments of the present technology. As illustrated in FIG. 14, raw barometer and IR data 1405 and 1410 can be collected at one or more fingertips. Monitoring operation 1415 can monitor for the initial physical contact between an object (e.g., a cup, steering wheel, weight, etc.) and the one or more fingertips based on the distance data 1410. Using the raw pressure and distance data, along with the initial contact data, generation operation 1420 can generate one or more biomimetic signals (e.g., FA1 1425, SA1 1430, FA2 1435, or the like). These signals can then be pushed to brain-machine interface 1440, neural interface 1445, or robotic interface 1450.

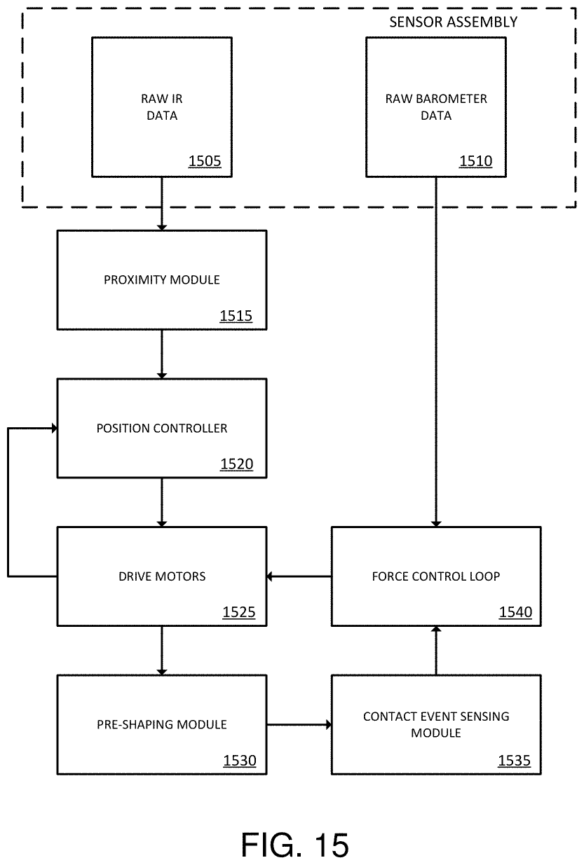

FIG. 15 is a block diagram illustrating a set of components that may be used to control a prosthetic hand or a robotic hand in accordance with various embodiments of the present technology. As illustrated in FIG. 15, raw barometer and IR data 1505 and 1510 can be collected from one or more sensor assemblies. The raw IR data can be fed into proximity module 1515, where positions of the sensor assemblies can be computed relative to the object. The position controller 1520 can generate one or more control signals to drive motors 1525. State information (e.g., current, voltage, position, etc.) from the drive motors can be feed back to the position controller 1520.

In some embodiments, pre-shaping module 1530 can pre-shape the hand causing the distance between each digit of the prehensor and the object to settle into the same constant distance. Contact detection module 1535 can detect the initial contact of each digit with the object (e.g., using the derivative of the IR sensor data 1505) and generate an indication of contact. This initial contact information along with the raw pressure data 1510 can be used by force control loop 1540 to set the pressure of each digit to a desired level.

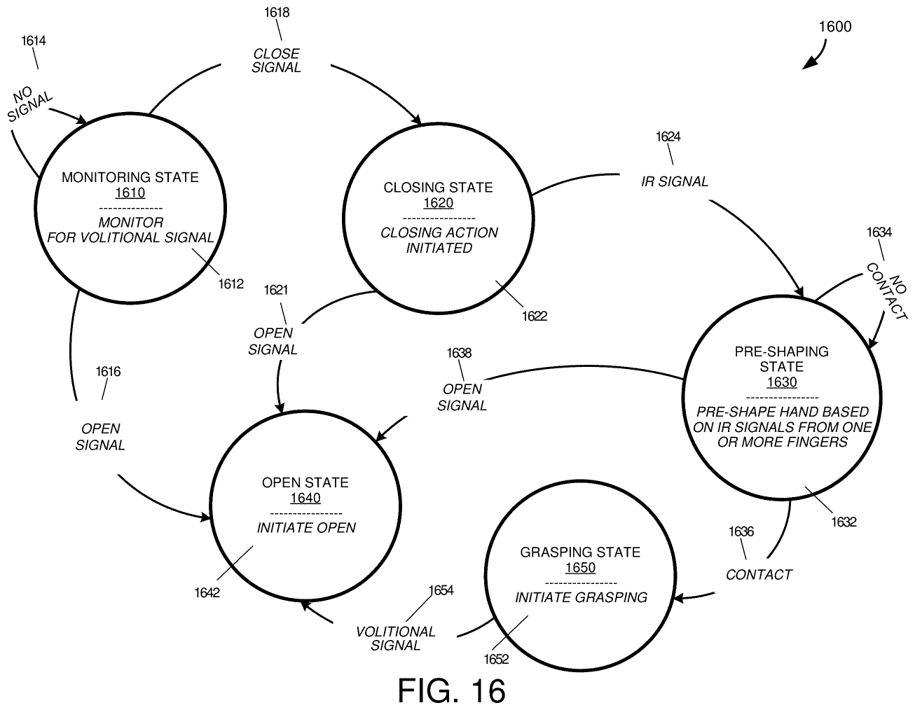

FIG. 16 is a state flow diagram 1600 illustrating an example of various operational states of a prosthetic hand or a robotic hand in accordance with various embodiments of the present technology. The natural approach to grasp can be broadly divided into three phases; i) object selection, ii) hand transport and pre-grasp shaping phase and finally, iii) grip phase. After the object of interest is chosen the hand approaches the object while simultaneously also pre-shaping according to the objects properties and anticipated use based on a priori knowledge. Finally, the grip phase involves final movement of the fingers touching the object for gentle pick up and manipulation thereafter.

Visual feedback provides a great deal of information about the environment and objects necessary for object selection, grasp planning and manipulation purpose. Tactile feedback on the other hand helps interpret the physical interactions of the object with the hand. Visual data however inherently suffers in low lightning conditions and occlusion form the hand itself. Hence, it is not suitable to accurately track the shape and position of the object during pre-grasp and grasp phase. Moreover, controlling a robot hand with high degree of freedom (DOF) is challenging given such inaccurate information from a vision sensor.

As such, some embodiments provide for a pre-grasp shaping of the robot hand using proximity sensors on the fingertips to reduce the complexity of controlling the hand to adapt to objects of varying sizes and shapes. Moreover, measuring the magnitude and location of contact eliminates the possibility of moving or damaging the object with imperfect contact forces. Various embodiments of the present technology create a reflex like behavior for pre-grasp and grasp phases using the upgraded design of the disclosed multimodal proximity-contact-force (PCF) sensor and a five-fingered robot hand using proximity signals for pre-grasp shaping and gently touching objects of unknown shapes with a five fingered robot hand.

As illustrated in FIG. 16, monitoring state 1610 executes event 1612 to monitor for a command signal. When no signal 1614 is detected, the system stays in monitoring state 1610. When open signal 1616 is detected, the system state transitions to open state 1640 where event 1642 is executed so that the motors are commanded to set the digits of the system to a predefined open state. When monitoring state 1610 detects a close signal 1618, the system transitions to closing state 1620 which executes event 1622 initiating a closing action by controlling the motors of the system. During closing state 1620, if an open signal 1621 is detected, the system will transition to open state 1640 described above. If however, an IR signal 1624 is detected, the system will transition from closing state 1620 to pre-shaping state 1630, where event 1632 causes the prehensor to pre-shape around the object based on the IR signals 1624 from the one or more sensor assemblies.

As long as no contact 1634 is detected the system will stay in pre-shaping state 1630. If a volitional open signal 1638 is detected the system will transition to open state 1640. If a contact signal 1636 is detected, then system will transition to grasping state 1650 where event 1652 initiates a grasping protocol causing the prehensor to grasp the object. If a volitional open signal 1654 is detected, the system will transition from grasping state 1650 to open state 1640.

An experimental setup was made of a five fingered Bebionic V2 prosthetic hand (RSL Steeper Inc.) equipped with the upgraded PCF sensors as shown in FIG. 1. The upgraded PCF sensor was a MEMS-based barometric pressure sensor (M55637-02BA03), in addition to the infrared proximity sensor (VCNL4040). In accordance with various embodiments, both were embedded inside an elastomer (rubber) layer. The resulting visual-haptic sensor can measure proximity, contact and force and also has the ability localize contact at eight discrete locations.

The robot hand had six DOFs. One DOF for each finger to open and close and one additional DOF in the thumb joint for abduction. The original electronics of the hand were replaced with a custom built motor controller boards from Sigenics Inc. The motor controller boards had an in-built PID position controller. The motors can also be driven by pulse width modulated (PWM) signal.

In order to measure the effectiveness of the embodiments implemented, a motion camera system was used to track the 6D pose of the object to provide an absolute change in its position before and after a grasp. Seven markers were attached on a cup and the makers were placed such that their 6D position is tracked by four cameras.

For pre-grasp shaping a simple Proportional Derivative and Integral (PID) controller was used to control the position of the fingers based on the proximity signals. However, other embodiments may use different controllers. Inputs to the controller were normalized proximity values from the PCF sensor and output of the controller is the Pulse Width Modulated (PWM) control signal for the finger motors. The PID gains were tuned for each of the fingers individually such that all fingers maintain a constant distance from an object.

In the contact detection phase the fingers are slowly moved towards the object with a constant PWM signal such that once contact is detected the finger motors are stopped. The contact was measured in the implemented embodiments by averaging (or smoothing) the raw proximity signal with an exponential averaging filter and subtract the original signal from this smoothed signal. Both the controllers were written in C programming language to avoid delays associated with transferring data over USB serial bus to the host computer. With this a decent response was obtained at around 100 Hz.

The experiment was started by placing a cup at a fixed location within the aperture of the hand. The performance of the both controllers (for pre-grasp shaping and contact) is tested against the case where no controller is used. A 10 g dead weight is placed in cup initially to balance the torque created by the markers. Weights are then incrementally added in the cup. Each trial consists of the hand initially fully open. An input from the experimenter sets the hand in the pre-grasp shaping mode. In this mode the fingers dynamically maintain a constant distant from the objects. Once all the fingers stop moving another input from the experimenter sets the hand in the grasp mode where the fingers gently move until contact is detected. Next trial starts by replacing the weight in the cup with next larger weight. The robot hand was fully opened and the cup was placed in the fixed location. Same steps were repeated for the case where no controller is used. In this case the robot fingers were position controlled to move to a set location where the cup is positioned.

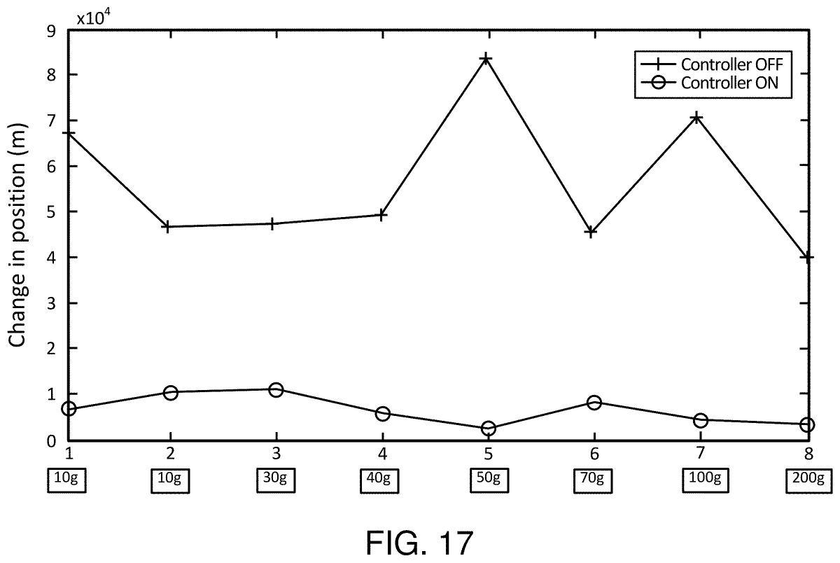

Data from the motion capture system was continuously recorded from the start of a trial until the fingers come in contact with the cup. A MATLAB function called findchangepts was used to find the point where there is a significant change in the signal. This function allowed for the calculation of the abrupt change in the resultant translational position of object by first averaging the position over the period of before and after the grasp and then subtracting them. This change in the resultant position of the cup with and without the use of the controller is shown in FIG. 17. It is clearly visible from the plot that when the controller is on the change in position of cup is significantly less compared to when the controller is off. A few outliers observed at dead weights 50 g, 70 g and 100 g were attributed to the poor repeatability of the movements of the robot fingers. This is the reason why the curves do not have a monotonically decreasing trend as the dead weight is increased.

Various embodiments provide a simple reflex behavior to pre-shape a five fingered robot hand and gently touch objects based on the proximity signals from the PCF sensor. Some embodiments may include a model or an on-the-fly calibration routine that would encode color dependence of the infrared proximity sensor. This would allow some embodiments to be extended to objects with any surface reflectively. Some embodiments may use a grip force control strategy in order to pick up objects with optimal force without damaging them. The motor friction of the robot fingers is not consistent across the entire range of the finger from fully open to fully close. Therefore, a single set of PID gains or PWM value for each finger does not allow the intended function of the finger. Sometimes it results in excessive motion while sometimes no motion at all. Some embodiments may address this issue by either using different set(s) of gains for different functional regions of the finger or by using some form of model predictive approach. Development of such a reflex like control of a multi-fingered robot hand is expected to dramatically improve grasp success and also help in effortless control of a upper limb prosthesis devices.

### CONCLUSION

Unless the context clearly requires otherwise, throughout the description and the claims, the words “comprise,” “comprising,” and the like are to be construed in an inclusive sense, as opposed to an exclusive or exhaustive sense; that is to say, in the sense of “including, but not limited to.” As used herein, the terms “connected,” “coupled,” or any variant thereof means any connection or coupling, either direct or indirect, between two or more elements; the coupling or connection between the elements can be physical, logical, or a combination thereof. Additionally, the words “herein,” “above,” “below,” and words of similar import, when used in this application, refer to this application as a whole and not to any particular portions of this application.

Where the context permits, words in the above Detailed Description using the singular or plural number may also include the plural or singular number respectively. The word “or,” in reference to a list of two or more items, covers all of the following interpretations of the word: any of the items in the list, all of the items in the list, and any combination of the items in the list.

The above Detailed Description of examples of the technology is not intended to be exhaustive or to limit the technology to the precise form disclosed above. While specific examples for the technology are described above for illustrative purposes, various equivalent modifications are possible within the scope of the technology, as those skilled in the relevant art will recognize. For example, while processes or blocks are presented in a given order, alternative implementations may perform routines having steps, or employ systems having blocks, in a different order, and some processes or blocks may be deleted, moved, added, subdivided, combined, and/or modified to provide alternative or subcombinations. Each of these processes or blocks may be implemented in a variety of different ways. Also, while processes or blocks are at times shown as being performed in series, these processes or blocks may instead be performed or implemented in parallel, or may be performed at different times. Further any specific numbers noted herein are only examples: alternative implementations may employ differing values or ranges.

The teachings of the technology provided herein can be applied to other systems, not necessarily the system described above. The elements and acts of the various examples described above can be combined to provide further implementations of the technology. Some alternative implementations of the technology may include not only additional elements to those implementations noted above, but also may include fewer elements.

These and other changes can be made to the technology in light of the above Detailed Description. While the above description describes certain examples of the technology, and describes the best mode contemplated, no matter how detailed the above appears in text, the technology can be practiced in many ways. Details of the system may vary considerably in its specific implementation, while still being encompassed by the technology disclosed herein. As noted above, particular terminology used when describing certain features or aspects of the technology should not be taken to imply that the terminology is being redefined herein to be restricted to any specific characteristics, features, or aspects of the technology with which that terminology is associated. In general, the terms used in the following claims should not be construed to limit the technology to the specific examples disclosed in the specification, unless the above Detailed Description section explicitly defines such terms. Accordingly, the actual scope of the technology encompasses not only the disclosed examples, but also all equivalent ways of practicing or implementing the technology under the claims.

To reduce the number of claims, certain aspects of the technology are presented below in certain claim forms, but the applicant contemplates the various aspects of the technology in any number of claim forms. For example, while only one aspect of the technology is recited as a computer-readable medium claim, other aspects may likewise be embodied as a computer-readable medium claim, or in other forms, such as being embodied in a means-plus-function claim. Any claims intended to be treated under 35 U.S.C. § 112(f) will begin with the words “means for”, but use of the term “for” in any other context is not intended to invoke treatment under 35 U.S.C. § 112(f). Accordingly, the applicant reserves the right to pursue additional claims after filing this application to pursue such additional claim forms, in either this application or in a continuing application.

1. A fingertip sensor comprising:
a proximity sensor to detect distance from the proximity sensor to an object and produce a proximity signal and detect an initial contact with the object,
wherein the proximity signal is sampled at a first rate;
a pressure sensor to detect pressure with the object and produce a pressure signal,
wherein the pressure signal is sampled at a second rate that is lower than the first rate;
a circuit with digital electronics to receive the proximity signal from the proximity sensor and the pressure signal from the pressure sensor and generate output signals indicative of a spatial relationship between the object and fingertip sensor,
wherein the spatial relationship includes an angular orientation between the object and the fingertip sensor; and
a viscoelastic compressible material enclosing the proximity sensor, the pressure sensor, and the circuit.

2. The fingertip sensor of claim 1, wherein the output signals identify spatial position of the object relative to the fingertip sensor.

3. The fingertip sensor of claim 1, wherein the proximity sensor includes an infrared (IR) emitter-detector to detect the distance and the pressure sensor includes a barometer to detect the initial contact with the object.

4. The fingertip sensor of claim 1, wherein the circuit includes a microprocessor configured to compute a derivative of the proximity signal and upon determining that the derivative of the proximity signal has exceeded a threshold generate an output signal that indicates contact with the object.

5. The fingertip sensor of claim 1, wherein the circuit includes:
a first analog to digital converter to produce a digitized proximity signal by sampling and quantizing the proximity signal;
a second analog to digital converter to produce a digitized pressure signal by sampling and quantizing the pressure signal;
a microprocessor communicably coupled to the first analog to digital converter and the second analog to digital convert and configured to receive the digitized proximity signal and the digitized pressure signal and produce an array of output signals indicative of a spatial position and an angular orientation of the object relative to the fingertip sensor; and
a communications module to transmit the array of output signals to a central controller board.

6. The fingertip sensor of claim 5, wherein the communications module uses an I²C protocol.

7. The fingertip sensor of claim 1, wherein the viscoelastic compressible material is formed from a liquid silicon polymer.

8. A method for operating an artificial hand having tactile sensors, the method comprising:
monitoring, using the tactile sensors, spatial position and angular orientation of an object relative to multiple tactile sensors;
transmitting the spatial position and angular orientation of the object relative to the tactile sensors to a central controller board; and
transitioning, based on commands from a central controller board, the artificial hand between multiple modes of operation, wherein the multiple modes of operation include:
an open mode of operation where the central controller board commands fingers of the artificial hand to extend to an open position;
a closing state of operation, entered from the open mode of operation upon receipt of a volitional signal, where the central controller board commands the fingers of the artificial hand to close around the object;
a pre-shaping state of operation, entered from the closing state of operation upon detection of a proximity signal exceeding a threshold, where the central controller board cause each finger to maintain an equal distance from the object while continuing to close; and
a grasping state of operation, entered from the pre-shaping state of operation upon detection of a contact signal exceeding a threshold, where the central controller board cause each finger to maintain a desired level of pressure.

9. The method of claim 8, wherein the volitional signal is a myoelectric signal collected from electrodes positioned on a limb of a subject.

10. An artificial prehensor comprising:
a plurality of tactile sensors, wherein each of the tactile sensors includes:
a proximity sensor to detect distance from the tactile sensor to an object and produce a proximity signal and detect contact with the object;
a pressure sensor to detect contact with the object and produce a pressure signal;
a circuit with digital electronics to receive the proximity signal from the proximity sensor and the pressure signal from the pressure sensor to identify spatial position and angular orientation of the object relative to the tactile sensor;
a viscoelastic compressible material enclosing the proximity sensor, the pressure sensor, and the circuit;
a central controller board configured to receive, from each of the tactile sensors, one or more signals representative of spatial position and angular orientation of an object relative to each of the tactile sensors and generate control signals; and
a set of actuators each configured to receive one or more of the control signals and set a position of a portion of the artificial prehensor.

11. The artificial prehensor of claim 10, wherein the central controller board generates a pre-shaping control signal based on the proximity signals produced by the tactile sensors.

12. The artificial prehensor of claim 11, wherein the controller includes a proportion, integral, and derivative (PID) controller with gains tuned for each finger to maintain a uniform distance from the object:

13. The artificial prehensor of claim 10, wherein the central controller board navigates through multiple modes of operations including:
an open mode of operation where the central controller board commands fingers of the artificial prehensor to extend to an open position;
a closing state of operation, entered from the open mode of operation upon receipt of a volitional signal, where the central controller board commands the fingers of the artificial prehensor to close around the object;
a pre-shaping state of operation, entered from the closing state of operation upon detection of the proximity signal exceeding a threshold, where the central controller board cause each finger to maintain an equal distance from the object while continuing to close; and
a grasping state of operation, entered from the pre-shaping state of operation upon detection of the contact signal exceeding a threshold, where the central controller board cause each finger to maintain a desired level of pressure.

14. The artificial prehensor of claim 13, further comprising a myoelectric interface to detect voluntary muscular contractions from a patient and generate the volitional signal.

15. The artificial prehensor of claim 10, wherein the control signals include pulse width modulated (PWM) control signals for each actuator in the set of actuators.

16. The artificial prehensor of claim 10, wherein the proximity sensor includes an infrared (IR) emitter-detector to detect the distance and the pressure sensor includes a barometer to detect the contact with the object.

17. The artificial prehensor of claim 10, wherein the circuit or the central controller board includes a microprocessor configured to compute a derivative of the proximity signal and upon determining that the derivative of the proximity signal has exceeded a threshold generate an output signal that indicates contact with the object.

18. The artificial prehensor of claim 10, wherein the circuit includes:
a first analog to digital converter to produce a digitized proximity signal by sampling and quantizing the proximity signal;
a second analog to digital converter to produce a digitized pressure signal by sampling and quantizing the pressure signal;
a microprocessor communicably coupled to the first analog to digital converter and the second analog to digital convert and configured to receive the digitized proximity signal and the digitized pressure signal and produce an array of output signals indicative of the spatial position and the angular orientation of the object relative to the tactile sensor; and
a communications module to transmit the array of output signals to the central controller board.

19. The artificial prehensor of claim 18, wherein the communications module uses an I²C protocol.

20. The artificial prehensor of claim 10, wherein the viscoelastic compressible material includes a liquid silicon polymer.

21. The artificial prehensor of claim 10, further comprising a machine learning engine to ingest the proximity signals and the pressure signals from the plurality of tactile sensors and generate an estimate of total force being applied to an object, position of forces applied to the object, and angular orientation of forces applied to the object.
